(19) United States
(12) Patent Application Publication
(10) Pub. No.: US 2024/0016749 A1
(43) Pub. Date: Jan. 18, 2024

(54) SOFT CAPSULE

(71) Applicant: SUNSHO PHARMACEUTICAL CO., LTD., Shizuoka (JP)

(72) Inventors: Kohei TOYOTA, Shizuoka (JP); Kenichi KOYAMA, Shizuoka (JP)

(73) Assignee: SUNSHO PHARMACEUTICAL CO., LTD., Shizuoka (JP)

(21) Appl. No.: 17/766,409

(22) PCT Filed: Sep. 25, 2020
(86) PCT No.: PCT/JP2020/036475

§ 371 Date: Apr. 4, 2022

(30) Foreign Application Priority Data
Oct. 11, 2019 (JP) 2019-188237

(51) Int. Cl. A61K 9/48

(52) U.S. Cl. CPC A61K 9/4816

(57) ABSTRACT

Provided is a novel soft capsule. The soft capsule has a shell comprising gellan gum, wherein the soft capsule satisfies the following requirements: (A) the soft capsule has a disintegration time of 60 minutes or less as measured by a disintegration test method specified in the Japanese Pharmacopoeia using water as a test medium; and/or (B) the soft capsule has a ratio of a crush strength (g) to an outer diameter. (mm) (crush strength/outer diameter ratio) of 210 or more.

### TECHNICAL FIELD

The present invention relates to a capsule (soft capsule). In particular, the present invention relates to a capsule that is used in the fields of medicine, food, industry, etc.

### BACKGROUND ART

Various materials such as gelatin are known as shell-forming base materials for soft capsules. The use of gellan gum as such a material has been attempted.

For example, Patent Literature 1 describes a seamless breakable capsule comprising a core and a shell, the shell comprising a gelling agent comprising gellan gum alone or in combination with another gelling agent, a filler, and a divalent metal ion sequestering agent.

### CITATION LIST

### Patent Literature

### Patent Literature 1: Japanese Patent No. 5529415

### SUMMARY OF INVENTION

### Technical Problem

As mentioned above, the use of gellan gum as a shell-forming base material has been attempted, but satisfactory results have not been obtained.

According to the present inventors' investigation, among shell materials, gellan gum seems to be difficult to use in terms of controlling capsule quality and physical properties (e.g., water disintegrability, oral disintegrability, etc.) as well as capsule manufacturing and production processes. Therefore, there are still many improvements to be made for the use of gellan gum as a capsule material.

In particular, in the case of the production of capsules that contain gellan gum as a shell material (e.g., capsules that contain substantially no or very little gelatin), a complicated step is required, such as immersion in an aqueous solution containing a hardening agent containing a divalent ion (e.g., calcium ion), and the water disintegrability of the produced capsules tends to be poor, as described in Patent Literature 1. This makes it very challenging to develop gellan gum-based capsules that can be efficiently produced and practically used.

An object of the present invention is to provide a novel capsule comprising gellan gum.

### Solution to Problem

After intensive studies to achieve the above-mentioned object, the present inventors found that, for example, a combination of a monovalent metal ion and gellan gum enables the production of a novel capsule and that such a capsule has excellent physical properties (e.g., achieving excellent water disintegrability, relatively high strength, or both of them although it comprises gellan gum). The present inventors conducted further investigation and then completed the present invention.

That is, the present invention relates to the following.

[1] A capsule (soft capsule) having a shell comprising gellan gum, wherein the soft capsule satisfies the following requirements :
(A) the soft capsule has a disintegration time of 60 minutes or less as measured by a disintegration test method specified in the Japanese Pharmacopoeia using water as a test medium; and/or
(B) the soft capsule has a ratio of a crush strength (g) to an outer diameter (mm) (crush strength/outer diameter ratio) of 210 or more, and
wherein the shell may be (substantially) free of gelatin,
[2] A capsule (soft capsule) having a shell comprising gellan gum and a monovalent metal ion, wherein the shell may be (substantially) free of gelatin.
[3] The capsule according to the above [1] or [2], wherein (A) the capsule has a disintegration time of 60 minutes or less as measured by a disintegration test method specified in the Japanese Pharmacopoeia using water as a test medium; and (B) the capsule has a ratio of a crush strength (g) to an outer diameter (mm) (crush strength/outer diameter ratio) of 210 or more.
[4] The capsule according to any one of the above [1] to [3], wherein (C) the capsule has a ratio of a crush deformation (mm) to an outer diameter (mm) (crush deformation/outer diameter ratio) of 0.1 or more.
[5] The capsule according to any one of the above [1] to [4], wherein the amount of the gellan gum in the shell is 5% by mass or more.
[6] The capsule according to any one of the above [1] to [5], wherein the monovalent metal ion is contained in the form of a monovalent metal compound.
[7] The capsule according to any one of the above [1] to [6], wherein the monovalent metal ion is contained in the form of at least one monovalent metal compound selected from alkali metal halides, alkali metal salts of organic acids, and alkali metal salts of sugars or polysaccharides.
[8] The capsule according to any one of the above [1] to [6], wherein the monovalent metal ion is contained at least in the form of an alkali metal salt of alginic acid.
[9] The capsule according to any one of the above [1] to [8], wherein the amount of the monovalent metal ion is 0.1 part by mass or more in terms of the mass of a metal atom relative to 100 parts by mass of the gellan gum.
[10] The capsule according to any one of the above [1] to [9], wherein the shell comprises an additional shell-forming base material in an amount of 100 parts by mass or less relative to 100 parts by mass of the gellan gum.
[11] The capsule according to any one of the above [1] to [10], wherein the shell comprises a plasticizer.
[12] The capsule according to any one of the above [1] to [11], wherein the shell comprises at least one plasticizer selected from polyhydric alcohols, sugar alcohols, disaccharides, polysaccharides, and derivatives thereof.
[13] The capsule according to any one of the above [1] to [12], wherein the capsule has an outer diameter of 0.1 to 15 mm.
[14] The capsule according to any one of the above [1] to [13], wherein the percentage of the shell in the capsule is 38 by mass or more.
[15] The capsule according to any one of the above [1] to [14], wherein the capsule has a content, and wherein the percentage of the shell in the capsule is 3 to 50% by mass.
[16] The capsule according to any one of the above [1] to [15], wherein the capsule has a crush strength with a standard deviation (SD) value of 500 g or less, and wherein the capsule has a crush deformation with a standard deviation (SD) value of 1 mm or less.
[17] The capsule according to any one of the above [1] to [16], wherein the capsule is a seamless capsule.
[18] A method for producing the capsule according to any one of the above [1] to [17], the method comprising at least the steps of:
forming a capsule by a drop method in such a manner that the capsule has a water content of 80% by mass or more and that the capsule has a ratio of a crush strength (g) to an outer diameter (mm) (crush strength/outer diameter ratio) of 5.0 or more; and drying the capsule obtained in the capsule formation step.

### Advantageous Effects of Invention

The present invention provides a novel capsule (soft capsule) comprising gellan gum.

Another embodiment of the present invention provides a capsule (soft capsule) that has excellent water solubility or excellent water disintegrability although it comprises gellan gum. In particular, such a capsule may be a capsule that is disintegrable within a relatively short time (e.g., within 60 minutes, in particular within 20 minutes) as measured, for example, in a disintegration test method specified in the Japanese Pharmacopoeia (17th revision).

In another embodiment, the capsule (soft capsule) of the present invention has a relatively high strength. For example, even when such a soft capsule has a content, the shell strength is relatively high.

In another embodiment, the capsule of the present invention achieves both excellent strength and excellent water disintegrability. According to the present inventors' investigation, capsule strength and water disintegrability are generally in a trade-off relationship in capsules comprising gellan gum, but in the present invention, appropriate selection of capsule composition, component proportions, etc., surprisingly, enables efficient production of capsules that achieves both of them.

In another embodiment, the capsule of the present invention can be efficiently produced although it comprises gellan gum. For example, the production of the capsule of the present invention does not need an immersion step described in Patent Literature 1. Cracking during capsule formation and drying steps can also be efficiently prevented. Therefore, the capsule of the present invention is advantageous also in terms of mass productivity etc.

### DESCRIPTION OF EMBODIMENTS

The capsule (soft capsule) of the present invention at least comprises gellan gum. In particular, the capsule may comprise gellan gum and a monovalent metal ion. The capsule usually comprises gellan gum (and other components such as a monovalent metal ion) in the shell (capsule shell). Specific examples of the soft capsule of the present invention include a capsule consisting of a monolayer sphere (consisting only of a shell) comprising gellan gum (and other components such as a monovalent metal ion) and a capsule having a shell comprising gellan gum (and other components such as a monovalent metal ion) (a capsule having a content and a shell comprising gellan gum and a monovalent metal ion).

Gellan gum is a straight-chain heteropolysaccharide produced by the non-pathogenic microorganism Pseudomonas elodea.

Types of gellan gum include a deacylated gellan gum and a native gellan gum. These can be used without any particular limitation in the present invention, but in terms of productivity of soft capsules, a deacylated gellan gum is preferable.

The deacylated gellan gum is a gellan gum in which the acyl groups (e.g., acetyl and glyceryl groups) of the native gellan gum are deacylated. Herein, the deacylated gellan gum may be a gellan gum in which all of the acyl groups are deacylated (fully deacylated gellan gum) or a gellan gum in which some of the acyl groups are deacylated (partially deacylated gellan gum).

The amount of the acyl group in the partially deacylated gellan gum is not particularly limited, and for example, the acylation degree, which is represented as the ratio of the peak intensity of the acyl group to the peak intensity of the methyl group measured (analyzed) by NMR, is 1.06 or less (e.g., less than 1.06, 1.05 or less, 1 or less, 0.95 or less, 0.9 or less, 0.8 or less, 0.7 or less, 0.6 or less, 0.5 or less, 0.4 or less, 0.3 or less, 0.2 or less, or 0.1 or less). The lower limit of the acylation degree only needs to be greater than 0. For example, it may be 0.001, 0.002, 0.003, 0.005, 0.007, 0.01, 0.015, 0.02, 0.025, 0.03, 0.035, 0.04, etc.

The amount of the gellan gum [or the amount of the gellan gum in the shell (or the amount of the gellan gum in the shell when the capsule has a content)] is not particularly limited. In order to improve the capsule strength etc., the amount of the gellan gum relative to the solids content [or relative to the total amount of the components other than the solvent such as water (shell components)] may be 5% by mass or more (e.g., 10% by mass or more, 15% by mass or more, 18t by mass or more), preferably 20% by mass or more; or may be 23% by mass or more, 25% by mass or more, 278 by mass or more, 30% by mass or more, or 33% by mass or more. The upper limit is not specified, and the amount of the gellan gum may be, for example, 80% by mass or less, 70% by mass or less, etc. to ensure easy handling during capsule formation and easy drying of capsules.

The capsule (shell) may comprise a monovalent metal ion. When the monovalent metal ion is used in the capsule (shell), it is easy to efficiently obtain a soft capsule with excellent physical properties (e.g., strength and water disintegrability).

Examples of the monovalent metal ion (or monovalent metal) include alkali metal ions (e.g., lithium, sodium, potassium, rubidium, cesium, or francium ion).

A single kind of monovalent metal ion or a combination of. two or more kinds of monovalent metal ions may be used.

The capsule may comprise a monovalent metal ion in the form of a monovalent ion-containing compound.

Examples of the compound include inorganic compounds [e.g., alkali metal halides (e.g., lithium chloride, sodium chloride, potassium chloride) etc.]; and organic compounds {e.g., alkali metal salts of organic acids (e.g., sodium acetate, sodium tartrate, sodium citrate, etc.), alkali metal salts of sugars or polysaccharides [e.g., alkaline metal salts of alginic acid (e.g., sodium alginate, potassium alginate) etc.], etc.}.

A single one of these compounds or a combination of two or more of them may be used.

Organic compounds (e.g., alkali metal salts of polysaccharides such as alkali metal salts of alginic acid) are preferable to inorganic compounds in view of capsule-forming efficiency. Among them, sodium alginate and potassium alginate are preferable, and in particular, sodium alginate is more preferable.

In some cases, the gellan gum originally contains an alkali metal (for example, in the form of an alkali metal salt with the carboxyl group that constitutes the gellan gum). Such an alkali metal shall not fall under the category of the “monovalent metal ion (monovalent metal)” in the present invention.

In other words, the “monovalent metal ion” in the present invention (capsule, shell) means a monovalent metal ion that is added or contained separately from the gellan gum [a monovalent metal (ion) that is not an alkali metal originally contained in the gellan gum] unless otherwise specified.

When the soft capsule comprises a monovalent metal ion, the amount of the monovalent metal ion for the amount of the monovalent metal ion in the shell [or the amount of the monovalent metal ion in the shell when the soft capsule has a content)] is not particularly limited. For example, the amount of the monovalent metal ion relative to the solids content [or relative to the total amount of the components other than the solvent such as water (shell components)] may be selected from the range of about 0.01% by mass or more (e.g., 0.03% by mass or more). The amount of the monovalent metal ion may be 0.05% by mass or more (e.g., 0.07% by mass or more), preferably 0.1% by mass or more, more preferably 0.3% by mass or more, in particular 0. 5% by mass or more [e.g., 0.8% by mass or more (e.g., 18 by mass or more, 1.15% by mass or more)], etc. The amount of the monovalent metal ion may be 30% by mass or less, preferably 20% by mass or less (e.g., 18% by mass or less), more preferably 15% by mass or less (e.g., 128 by mass or less), in particular 10% by mass or less (e.g., 8% by mass or less, 5% by mass or less), etc.

When the capsule comprises a monovalent metal ion in the form of a compound, the amount of the monovalent metal compound [or the amount of the monovalent metal compound in the shell (or the amount of the monovalent metal compound in the shell when the capsule has a content)] can be selected as appropriate according to the type of compound, the amount (concentration) of the monovalent metal in the compound, etc. For example, the amount of the monovalent metal compound relative to the solids content [or relative to the total amount of the components other than the solvent such as water (shell components)] may be selected from the range of about 0.01% by mass or more (e.g., 0.03% by mass or more). The amount of the monovalent metal compound may be 0.1% by mass or more (e.g., 0.15% by mass or more), preferably 0.2% by mass or more, more preferably 0.5% by mass or more, in particular 18 by mass or more [e.g., 1.5% by mass or more (e.g., 2% by mass or more, 2.5% by mass or more, 3% by mass or more)], etc. The amount of the monovalent metal compound may be 80% by mass or less (e.g., 70% by mass or less), preferably 60% by mass or less (e.g., 55% by mass or less), more preferably 508 by mass or less (e.g., 45% by mass or less), in particular 40% by mass or less (e.g., 35% by mass or less, 30% by mass or less, 25% by mass or less, 20% by mass or less), etc.

In particular, when the capsule comprises a monovalent metal ion in the form of an organic compound (e.g., an alkali metal salt of a sugar or polysaccharide such as an alkali metal salt of alginic acid), the amount of the compound relative to the solids content [or relative to the total amount of the components other than the solvent such as water (shell components)] may be 0. 1% by mass or more, preferably 18 by mass or more, more preferably 5$ by mass or more, in particular 10% by mass or more, etc. The amount of the compound may be 808 by mass or less (e.g., 70% by mass or less), preferably 60% by mass or less (e.g., 55% by mass or less), more preferably 50% by mass or less (e.g., 45% by mass or less), in particular 40% by mass or less, etc.

When the capsule comprises a monovalent metal ion, the amount of the monovalent metal ion may be, for example, 0.01 part by mass or more (e.g., 0. 05 part by mass or more), preferably 0.1 part by mass or more (e.g., 0.3 part by mass or more), more preferably 0.5 part by mass or more (e.g., 0.8 part by mass or more), in particular 1 part by mass or more (e.g., 1.5 parts by mass or more, 2 parts by mass or more, 2.5 parts by mass or more, 3 parts by mass or more), etc. relative to 100 parts by mass of the gellan gum. The amount of the monovalent metal ion may be 100 parts by mass or less (e.g., 80 parts by mass or less), preferably 50 parts by mass or less (e.g., 40 parts by mass or less), more preferably 30 parts by mass or less (e.g., 25 parts by mass or less), etc. ; or may be 20 parts by mass or less (e.g., 18 parts by mass or less, 15 parts by mass or less, etc.), etc. relative to 100 parts by mass of the gellan gum.

When the capsule comprises a monovalent metal ion in the form of a compound, the amount of the monovalent metal compound may be, for example, 0.1 part by mass or more (e.g., 0.5 part by mass or more), preferably 1 part by mass or more (e.g., 2 parts by mass or more), more preferably 3 parts by mass or more (e. g., 4 parts by mass or more), in particular 5 parts by mass or more (e.g., 7 parts by mass or more, & parts by mass or more, 10 parts by mass or more), etc. relative to 100 parts by mass of the gellan gum. The amount of the monovalent metal compound may be 500 parts by mass or less (e.g., 400 parts by mass or less), preferably 300 parts by mass or less (e.g., 250 parts by mass or less), more preferably 200 parts by mass or less (e.g., 150 parts by mass or less), etc.; or may be 120 parts by mass or less (e.g., 100 parts by mass or less, 90 parts by mass or less, 80 parts by mass or less, 70 parts by mass or less, etc.), etc. relative to 100 parts by mass of the gellan gum.

In particular, when the capsule comprises a monovalent metal ion in the form of an organic compound (e.g., an alkali metal salt of a sugar or polysaccharide such as an alkali metal salt of alginic acid), the amount of the compound may be, for example, 0.1 part by mass or more (e.g., 0.5 part by mass or more), preferably 1 part by mass or more (e.g., 3 parts by mass or more), more preferably 5 parts by mass or more (e.g., 8 parts by mass or more), in particular 10 parts by mass or more (e.g., 15 parts by mass or more, 20 parts by mass or more, 25 parts by mass or more, 30 parts by mass or more), etc. relative to 100 parts by mass of the gellan gum. The amount of the compound may be 500 parts by mass or less (e.g., 400 parts by mass or less), preferably 350 parts by mass or less (e.g., 300 parts by mass or less), more preferably 250 parts by mass or less (e.g., 200 parts by mass or less), etc.; or may be 150 parts by mass or less (e.g., 120 parts by mass or less, 100 parts by mass or less), etc. relative to 100 parts by mass of the gellan gum.

When the amount of the monovalent metal ion (compound) used is as described above, it is easy to efficiently obtain a capsule that is desirable in terms of disintegrability, capsule strength, etc.

In addition to the monovalent metal ion, the capsule (or the shell) of the present invention may or may not comprise a multivalent metal ion (e.g., a divalent metal ion such as a calcium ion). When the capsule of the present invention comprises a multivalent metal ion, its amount (its amount in the shell) is not particularly limited, but is preferably relatively small. For example, the amount of the multivalent metal ion (or multivalent metal) may be 5 parts by mass or less, preferably 3 parts by mass or less, more preferably 1 part by mass or less, even more preferably 0.1 part by mass or less, etc. relative to 100 parts by mass of the monovalent metal ion (or the monovalent metal).

In some cases, the gellan gum originally contains a multivalent metal ion (for example, in the form of an alkaline earth metal salt with the carboxyl group that constitutes the gellan gum). Such a multivalent metal shall not fall under the category of the “multivalent metal ion (multivalent metal)” described above.

In the capsule (shell), the gellan gum may be combined with an additional shell-forming base material, if necessary. The additional shell-forming base material is not particularly limited, and examples include gelatin, carrageenan, agar, pectin, locust bean gum, xanthan gum, guar gum, tara gum, welan gum, tamarind seed gum, gum ghatti, psyllium seed gum, tragacanth gum, linseed gum, diutan gum, gum arabic, curdlan, furcellaran, pullulan, glucomannan, alginic acid, degradation products thereof (e.g., hydrolyzed guar gum, etc.), and salts thereof.

In particular, the capsule [the shell (the shell-forming base material) that constitutes the capsule] is preferably a plant-based one (mainly composed of plant components). In this view, when the additional shell-forming base material is used, it is preferable that the additional shell-forming base material (or the shell) is a plant-based one (mainly composed of plant components). Typically, the capsule [the shell (the shell-forming base material) that constitutes the capsule] is preferably free (substantially free) of animal components (e.g., gelatin etc.).

The additional shell-forming base material may be used as a monovalent metal ion-containing component (a monovalent metal compound). For example, the alkali metal salt of alginic acid described above can be used also as the additional shell-forming base material.

A single kind of additional shell-forming base material or a combination of two or more kinds of additional shell-forming base materials may be used.

When the additional shell-forming base material is used, its amount is not particularly limited and is determined according to the type of additional shell-forming base material etc., but in particular, the composition of the shell may be determined such that the gellan gum is a main shell-forming base material. For example, the amount of the additional shell-forming base material is 200 parts by mass or less (e.g., 150 parts by mass or less), preferably 100 parts by mass or less, more preferably 80 parts by mass or less (e.g., 60 parts by mass or less, 50 parts by mass or less, 40 parts by mass or less, 30 parts by mass or less, 20 parts by mass or less), etc. relative to 100 parts by mass of the gellan gum. When the additional shell-forming base material is used, the lower limit is not specified and may be, for example, 0.1 part by mass, 0.5 part by mass, 1 part by mass, 2 parts by mass, 3 parts by mass, 5 parts by mass, 8 parts by mass, 10 parts by mass, 12 parts by mass, 15 parts by mass, etc. relative to 100 parts by mass of the gellan gum.

As mentioned above, the additional shell-forming base material is preferably a plant-based material. In this view, when the additional shell-forming base material contains an animal component (e.g., gelatin etc.), the amount of the animal component (e.g., gelatin etc.) may be about 10 parts by mass or less or substantially zero relative to 100 parts by mass of the gellan gum [the amount of the animal component may be 5 parts by mass or less (e.g., 3 parts by mass or less, 2 parts by mass or less, 1 part by mass or less, 0 part by mass) relative to 100 parts by mass of the gellan gum].

The capsule (shell) may further comprise a plasticizer to adjust the strength of the shell, for example. Any known plasticizer in the art can be used without any particular limitation. Specific examples of the plasticizer include polyhydric alcohols (e.g., (poly) alkylene glycols such as ethylene glycol, propylene glycol, polyethylene glycol, and polypropylene glycol; and polyols with three hydroxyl groups or more, such as glycerin); sugars {e.g., monosaccharides (e.g., glucose, fructose, glucose, galactose, etc.), disaccharides (e.g., sucrose, maltose, trehalose, coupling sugar, etc.), oligosaccharides (e.g., maltooligosaccharides etc.), sugar alcohols (e.g., sorbitol, maltitol, lactitol, hydrogenated isomaltulose, xylitol, mannitol, galactitol, erythritol, reducing syrup, etc.), and polysaccharides or derivatives thereof [e.g., starch, starch derivatives (e.g., polydextrose, dextrin, maltodextrin, indigestible dextrin, cyclodextrins (α, β, and γ), etc.), cellulose derivatives (e.g., hydroxypropylcellulose, hydroxypropylmethylcellulose, methylcellulose, carboxymethylcellulose, etc.), etc.]}; polyvinyl alcohol; and triacetin.

A single one of these plasticizers or a combination of two or more of them may be used.

Among these examples, polyhydric alcohols, sugar alcohols, disaccharides, polysaccharides, and derivatives thereof (e.g., polysaccharide derivatives such as starch derivatives, cellulose derivatives, etc.) are preferred because they provide the capsule with elasticity after drying, improve water disintegrability, improve the tactile feel or texture of the shell, improve the workability (handleability) of the shell-forming liquid, and increase the solids concentration of the shell-forming liquid, which results in a shorter drying time of the capsule. Among them, glycerin, propylene glycol, sorbitol, erythritol, trehalose, starch, starch derivatives, and cellulose derivatives are more preferred. For example, when the plasticizer is contained in the shell, the amount of the plasticizer in the shell is not particularly limited and can be, for example, 10 to 50% by mass.

In addition to the aforementioned components, the capsule (shell) can comprise other components known in the art. Such components include, for example, colorants (dyes, pigments), flavors, sweeteners, antioxidants, preservatives, seasonings, spices, acidulants (e.g., citric acid or salts thereof), bittering substances, salts, umami components, and other components described in the later section on the capsule content (e.g., physiologically active substances, biologically active substances, etc.). A single one of these components of a combination of two or more of them may be used. Some types of sweeteners can be used also as the shell-forming base material or the plasticizer described above. The amount of the components mentioned in this section is not particularly limited and may be, for example, 50% by mass or less.

The capsule may consist only of a shell, as described above, or consist of a shell and a content. For a capsule having a content, the capsule content is particularly limited. The form of the capsule content is not particularly limited and may be, for example, liquid, solid, or in the form of coexistence thereof.

Specific examples of the capsule content include fragrances, cosmetics, surfactants, detergents, bath salts, coolants, physiologically active substances (e.g., vitamins, amino acids, collagen, collagen peptides, lipids (oils and fats [oils (e.g., medium-chain triglyceride oil (MCT), olive oil, fish oil, linseed oil, etc.) and fats (e.g., butter, margarine, coconut oil, shea butter, etc.)], waxes, etc.), isoflavones, minerals, enzymes, hormones, etc.), biologically active substances (e.g., medicines), microorganisms (e.g., bacteria such as lactic acid bacteria, bifidobacteria, natto bacteria, and yeasts; fungi such as yeasts; etc.), foods and drinks (or extracts thereof), plants (or extracts thereof), sweeteners, acidulants, seasonings, and revitalizers.

A single one of these substances or a combination of two or more of them may be used.

The amount of the capsule content is not particularly limited and can be set as appropriate according to the size and application of the capsule. For example, the mass ratio of the capsule content to the shell may be one or more, with the shell being one.

The capsule (soft capsule) may be a seamless capsule. A typical example of the capsule (soft capsule) is a seamless capsule having a content and a shell.

The shape of the capsule is not particularly limited and may be, for example, a sphere (e.g., a complete sphere) or a football-like shape.

The outer diameter of the capsule is not particularly limited and may be 0.1 mm or more, 0.5 mm or more, 1 mm or more, 1.5 mm or more, 2 mm or more, etc. The outer diameter of the capsule may be 30 mm or less, 25 mm or less, 20 mm or less, 18 mm or less, 15 mm or less, 12 mm or less, 10 mm or less, 8 mm or less, etc. Typically, the outer diameter of the capsule may be 0.1 to 20 mm (e.g., 0.1 to 15 mm), 1 to 15 mm, 1.5 to 10 mm, 2 to 8 mm, etc. The outer diameter refers to the major axis when the horizontal plane (cross-section) of the capsule is circular, and to the maximum diameter when it is not circular.

The outer diameter can be measured using, for example, a digital caliper (trade name: Quick Mini 25, model number: PK-0510SU, measurement range: 0 to 25 mm, manufactured by Mitutoyo Corporation).

When the capsule has no contents (or consists only of a shell), the outer diameter is the diameter of the shell. The outer diameter may be a value measured under predetermined conditions (e.g., 40 to 60% RH, 45% RH, etc.). The same applies to the physical properties (e.g., shell thickness, crush strength, elasticity, etc.) of the soft capsule unless otherwise noted with “before drying”.

When the capsule has a content, the thickness of the capsule shell is not particularly limited and may be, for example, about 5 to 120 μm, about 10 to 100 μm, about 20 to 90 μm, or about 20 to 60 μm.

The thickness (depth) of the shell can be measured using, for example, a digital microscope (trade name; VHX-900, manufactured by Keyence Corporation).

When the capsule has a content, the mass ratio of the capsule content to the shell can be selected according to the thickness (outer diameter) of the capsule etc. and is not particularly limited. For example, the mass ratio of the capsule content to the shell can be selected from the range of about 1 part by mass or more (e. g., 10 parts by mass or more), preferably about 30 parts by mass or more (e.g., 50 parts by mass or more), more preferably about 60 parts by mass or more (e.g., 100 parts by mass or more) relative to 100 parts by mass of the shell. The mass ratio of the capsule content to the shell may be 4000 parts by mass or less (e.g., 3500 parts by mass or less), preferably 3000 parts by mass or less (e.g., 2500 parts by mass or less), more preferably 2000 parts by mass or less, etc. relative to 100 parts by mass of the shell.

The percentage of the shell in the capsule is not particularly limited and may be, for example, 18 by mass or more, 2% by mass or more, 3% by mass or more, etc. When the capsule has a content, the upper limit of the percentage of the shell in the capsule (the percentage of the mass of the shell relative to the combined total mass of the shell and the capsule content) is not specified and may be, for example, 80% by mass, 70% by mass, 60% by mass, 50% by mass, 40% by mass, 35% by mass, 30% by mass, 25% by mass, 22% by mass, 20% by mass, 18% by mass, etc.

In particular, the percentage of the shell in the capsule having a content may be, for example, about 1 to 50% by mass (e.g., 2 to 408 by mass), preferably about 3 to 20% by mass, more preferably about 3 to 18% by mass; or may be usually 3 to 50% by mass (e.g., about 3 to 40% by mass).

When the capsule has no contents (or consists only of a shell), the percentage of the shell in the capsule is 100% by mass. Obviously, capsules having no contents (capsules with the percentage of the shell being 100%) are also included in the present invention.

The water content of the capsule (the water content of the shell) can be set as appropriate according to the application of the capsule etc. For example, the water content may be 30% by mass or less, 25% by mass or less, or 20% by mass or less. The water content may be 18 by mass or more, 3% by mass or more, 5% by mass or more, 8% by mass or more, etc. For practical use, the water content may be 10 to 18% by mass, etc.

The water content can be determined by any conventional method [e.g., by loss-on-drying measurement (a method based on the mass before and after drying)].

The capsule of the present invention is highly likely to achieve sufficient water disintegrability (excellent water disintegrability). In this case, the capsule can have a relatively short disintegration time (e.g., within 60 minutes) as measured in a disintegration test method specified in the Japanese Pharmacopoeia (17th revision) using water (37±2° C.) as the test medium. The disintegration time may be within 40 minutes, within 30 minutes, etc., and in particular within 20 minutes, within 10 minutes, etc. The lower limit of the disintegration time is not specified and may be, for example, 30 seconds, 1 minute, 2 minutes, 3 minutes, 4 minutes, 5 minutes, etc. In the disintegration test described above, auxiliary disks can be used if necessary.

The crush strength of the capsule is determined according to the outer diameter etc. and may be, for example, 100 g or more, 200 g or more, 300 g or more, 400 g or more, 500 g or more, 600 g or more, 700 g or more, 800 g or more, 900 g or more, 1000 g or more, etc.

The upper limit of the crush strength of the capsule is not specified, and the crush strength of the capsule may be, for example, 20000 g or less, 15000 g or less, 12000 g or less, 10000 g or less, etc.

The crush strength can be measured using, for example, a rheometer (CR-3000EX, manufactured by Sun Scientific).

The ratio of the crush strength (g) to the outer diameter (mm) (crush strength/outer diameter ratio) of the capsule (e.g., the capsule having a content) is not particularly limited and may be, for example, 200 or more (e.g., more than 200), preferably 210 or more (e.g., 220 or more), more preferably 230 or more (e.g., 240 or more) ; or may be 250 or more, 300 or more, 400 or more, etc.

The upper limit of the ratio of the crush strength to the outer diameter (crush strength/outer diameter ratio) is not specified and may be, for example, 20000, 15000, 10000, 8000, 6000, 5000, etc.

The ratio of the crush strength to the outer diameter can be said to be an index that reflects the practical breakability of the capsule, because there can be cases where the capsule is easily breakable even when the crush strength is high (for example, a case where the outer diameter is long).

The crush deformation of the capsule is determined according to the outer diameter etc. and may be, for example, 0.1 mm or more, 0.2 mm or more, 0.5 mm or more, 1.0 mm or more, etc.

The upper limit of the crush deformation of the soft capsule is not specified, and the crush deformation e of the soft capsule may be, for example, 15 mm or less, 10 mm or less, 8 mm or less, etc.

The crush deformation can be measured using, for example, a rheometer (CR-3000EX, manufactured by Sun Scientific).

The ratio of the crush deformation (mm) to the outer diameter (mm) (crush deformation/outer diameter ratio) in the capsule is not particularly limited and may be, for example, 0. 1 or more, preferably 0,12 or more, more preferably 0.15 or more; or may be 0.18 or more, 0.2 or more, etc.

The upper limit of the ratio of the crush deformation to the outer diameter (crush deformation/outer diameter ratio) is not specified and may be, for example, 1.0, 0.98, 0.97, 0.96, 0.95, etc.

The ratio of the crush deformation to the outer diameter can be said to represent the degree of elasticity (ability to deform until the capsule crushes). Thus, this ratio can be said to be a supplementary index that reflects the strength (breakability) of the capsule.

The capsule of the present invention is highly likely to have such a relatively high (or sufficient) mechanical strength.

In a particularly preferable embodiment, the capsule of the present invention can have both such mechanical strength and the above-described water disintegrability although it comprises gellan gum.

In general, thicker capsule shells (a higher percentage of the shell in the capsule) are required to ensure that the capsules can endure the production process (in particular, the drying step), but thicker shells tend to cause poor water disintegrability. In other words, it is challenging to achieve both sufficient mechanical strength and sufficient disintegrability in capsules. According to the present inventors' investigation, this tendency was particularly marked in capsules comprising gellan gum in the shell.

However, according to the present invention, both of them can be efficiently achieved by adjustment of the shell formula etc.

Efficient production of the capsules having both of the properties can be achieved by appropriate selection of components to be combined with the gellan gum (e.g., other components such as a monovalent metal ion or a monovalent metal compound, an additional shell-forming base material, a plasticizer, etc.), their proportions, etc. (as well as capsule production conditions).

In the present invention, a number of relatively uniform Capsules can be obtained.

The standard deviation (SD) value of the crush strength of the capsules is determined according to the crush strength value etc. and may be, for example, 800 g or less, 700 g or less, 600 g or less, 500 g or less, 400 g or less, 350 g or less, etc.

The standard deviation (SD) value of the crush deformation of the capsules is determined according to the crush strength value etc. and may be, for example, 2 mm or less, 1. 5 mm or less, 1 mm or less, 0.8 mm or less, 0.7 mm or less, 0.6 mm or less, 0.5 mm or less, etc.

The capsule (soft capsule) can be produced according to known methods. For example, the capsule can be produced by a drop method [a drop method using a nozzle (e.g., a multi-nozzle)] using a liquid containing capsule (shell)-forming components (e.g., gellan gum and other components such as a monovalent metal compound, an additional shell-forming base material, a plasticizer, etc.) (a Shell-forming liquid).

In general, the shell-forming liquid often contains water as a solvent component.

The solids concentration in the shell-forming liquid may be, for example, 3% by mass or more (e.g., 4% by mass or more), preferably 5% by mass or more, and more preferably 62 by mass or more. The solids concentration may be 20% by mass or less (e.g., 15% by mass or less), preferably 12% by mass or less, more preferably 10% by mass or less, etc.

The production conditions can be set as appropriate according to the composition of the capsule, the dimension of the capsule diameter, the percentage of the shell in the capsule, etc. Similarly, the cooling temperature, cooling time, drying temperature, and drying time after dropping can also be set to appropriate values.

The capsule formed by a drop method may be immersed in an immersion liquid (e.g., a liquid containing a multivalent metal ion such as a calcium ion) before drying, but according to the present invention, the capsule (shell) composition including the above-mentioned components etc. allows elimination of the need for such immersion that can impair water disintegrability.

The present invention facilitates efficient gelation while imparting adequate strength to the gellan gum. This makes it easy to efficiently obtain a capsule (soft capsule) that has adequate strength to endure the production process and yet has excellent physical properties (e.g., water disintegrability and mechanical strength).

Thus, the capsule of the present invention can be efficiently produced by the above-mentioned drop method, etc. Typically, the capsule can be produced by at least the following steps: a capsule formation step to form a capsule (capsule with high water content) by a drop method and a drying step to dry the capsule obtained in the capsule formation step.

In such a series of steps, in particular, appropriate setting of the conditions before drying (for a capsule before the drying step) can prevent deformation, cracking, and other defects due to drying, thereby facilitating efficient production.

The water content before drying (the water content of the shell in the capsule before drying) is usually high and may be, for example, 70% by mass or more, 75% by mass or more, 80% by mass or more, 85% by mass or more, 908 by mass or more, etc. The water content before drying may be 99% by mass or less, 98% by mass or less, 97% by mass or less, 96% by mass or less, 95% by mass or less, etc.

The outer diameter of the capsule before drying is not particularly limited and is usually 0.1 mm or more (e.g., 1 to 20 mm). The outer diameter of the capsule before drying may be 0.75 to 22.5 mm, 25 mm or less (e.g., 0.5 to 25 mm), etc.

The outer diameter can be measured using, for example, a digital caliper (trade name: Quick Mini 25, model number: PK-0510SU, measurement range: 0 to 25 mm, manufactured by Mitutoyo Corporation).

The crush strength of the capsule before drying is determined according to the outer diameter and cannot be definitely specified, but it is usually 10 g or more (e.g., 50 to 1000 g) and may be 30 to 1500 g, 3000 g or less (e.g., 10 to 3000 g), etc. The crush strength can be measured using a rheometer (CR-3000EX, manufactured by Sun Scientific).

The ratio of the crush strength (g) to the outer diameter (mm) (crush strength/outer diameter ratio) of the capsule before drying may be 3 or more, 3.5 or more, 4 or more, or 4.5 or more, and in particular 5.0 or more. When the ratio is greater than or equal to the aforementioned value, the capsule obtained after drying has adequate strength to endure the production process and yet is disintegrable in water. The crush strength/outer diameter ratio may be, for example, 5.5 or more ox 6. 0 or more to prevent the capsule from cracking due to drying. The upper limit is not specified, and the crush strength/outer diameter ratio may be, for example, 15 or less.

The capsule of the present invention can be produced by the production method described above as well as other known production methods such as the method described in Japanese Patent No. 5047285 or Japanese Patent Application Publication No. H10-506841. Specifically, a drop method using a double or multiple (triple or more) nozzle (a method for producing a seamless capsule) can be used. An exemplary production method includes the step of loading a capsule content into a capsule shell comprising gellan gum (and other components such as a monovalent metal ion) to prepare a loaded capsule. After the preparation of the loaded capsule, the shaping and drying steps may be included. The capsule content may have the same composition as that of the capsule shell-forming liquid and may be determined with reference to the previous section on the capsule of the present invention.

The drop method uses, for example, a composite nozzle device in which nozzles are properly aligned in an approximately concentric manner. The composite nozzle device has, for example, an inner nozzle for receiving and dispensing the capsule content and an outer nozzle for receiving and dispensing the capsule shell-forming liquid, and the inner and outer nozzles are aligned concentrically. With the composite nozzle device, the capsule content is discharged from the outlet of the inner nozzle, and the capsule shell-forming liquid is discharged from the outlet of the outer nozzle. The capsule content and the capsule shell-forming liquid are simultaneously discharged into an oil or gas from the inner and outer nozzles, respectively, at a constant speed by a pump or gravity to form a coaxial flow in the downstream carrier flow. To the flow, a physical force such as vibration is applied to cut the discharged liquid at regular intervals. The cut portions are formed into spheres by the interfacial or surface tension between the oil or gas and the capsule shell-forming liquid, and the shell layer is gelled by cooling to form capsules.

The interfacial or surface tension is not particularly limited. For example, the interfacial tension between the capsule shell-forming liquid and the capsule content is preferably 15 to 50 mN/m. The interfacial or surface tension can be measured using, for example, Sigma 702 manufactured by KSV INSTRUMENTS (FINLAND).

In the present invention, it is preferable that the temperature conditions in the vicinity of the multi-nozzle are appropriately controlled during the capsule formation. For example, the temperature in the vicinity of the multi-nozzle of the capsule (seamless capsule) forming machine is preferably set to the temperature range described below.

(1) The temperature of the capsule content is controlled to a set point of ±2° C. (more preferably ±1° C.) within a predetermined temperature range in which the capsule content is liquid (or flowable). The predetermined temperature range is determined according to the properties of the capsule content. For example, (i) when the capsule content is liquid (at ordinary temperature), the predetermined temperature range is 5 to 25° C. (more preferably 12 to 22° C.); (ii) when the capsule content is solid at ordinary temperature, the predetermined temperature range is a temperature range in which they are kept liquid [for example, when fats (e.g., butter, margarine, or others that are at room temperature at 40° ° C. or lower) are used, the predetermined temperature range is 30 to 60° C. (preferably 40 to 50° C.) etc.]; etc.
(2) The temperature of the capsule shell-forming liquid is controlled to a set point of ±2°0 C. (preferably ±1° C.) within a range of 50 to 99° C. (preferably 60 to 95° C.).

When a lipophilic solvent is used in combination with an oily component of the capsule contents, (3) the temperature of the lipophilic solvent is preferably controlled to a set point of ±1° C. (preferably : 0.5° C.) within a range of 1 to 25° C. (more preferably 5 to 20° C.).

In addition to the above conditions, (4) the difference between the temperature of the capsule content and the temperature of the capsule shell-forming liquid is preferably 25 to 94° C. (more preferably 38 to 85° C.).

When a lipophilic solvent is used in combination with an oily component of the capsule contents, (5) the difference between the temperature of the capsule shell-forming liquid and the temperature of the lipophilic solvent is preferably 35 to 94° C. (more preferably 49 to 85° C.).

After passing through the nozzle, the shell layer is cooled and gelled in a cooling oil. The cooling temperature to be achieved using the cooling oil is, for example, about 5 to 35° C.

Those skilled in the art would appreciate that the above temperature conditions (1) to (5) can be used alone or in combination as appropriate according to the quality level required for the capsule (seamless capsule). Those skilled in the art would appreciate that the above temperature control can be easily performed by, for example, a combination of PID control and feedback control, but the control method is not limited thereto.

There is no particular limitation on the method for preparing the capsule shell-forming liquid containing gellan gum (and if necessary, other components such as a monovalent metal ion). For example, the capsule shell-forming liquid can be prepared by dissolving the component (s) in a solvent. The solvent is not particularly limited as long as it does not interfere with the effects of the present invention. Examples of the solvent include water, ethanol, and other alcohols. Preferred is water.

Furthermore, when the formed capsule (s) are cooled, the cooling temperature is not particularly limited and is usually 20° C. or less, preferably 10° C. or less. The cooling time is not particularly limited and usually ranges from about 10 minutes to about 30 hours.

After wet capsules are formed by the above procedure, they are dried. The drying is generally performed using, for example, a “rotary drum dryer” equipped with a ventilation device. For smaller capsules such as seamless capsules, a fluidized dryer can be used, in which the capsules are dried while they are blown up and fluidized. The drying temperature is not particularly limited and may range from about 20 to 50° C.

### EXAMPLES

Hereinafter, the present invention will be described in detail by examples, but the present invention is not limited thereto.

The produced capsules were subjected to measurement or evaluation according to the following methods.

### Capsule Crush Strength and Elasticity (Crush Deformation)

The crush strength of the capsules (before and after drying) was measured using a rheometer CR-3000EX manufactured by Sun Scientific at room temperature (22 to 27° C.) and 40 to 60% RH.

The strength of the capsules after drying was measured after they had been left in a 45% RH environment for a sufficient period of time (about half a day) (the water content was about 10 to 188 by mass). Hereinafter, the same applies to the capsules after drying.

In the above measurement, the distance by which the capsule deformed until it crushed (the distance by which the capsule was compressed with the rheometer until the capsule crushed) was used as an index of the elasticity of the capsule.

In addition, the standard deviation (SD) values of the crush strength and the crush deformation were calculated for n=20.

### Capsule Outer Diameter

The outer diameter of the capsules was measured using a digital caliper manufactured by Mitutoyo Corporation (trade name: Quick Mini 25, model number: PK-0510SU, measurement range: 0 to 25 mm) at room temperature (22 to 27° C.) and 40 to 60% RH.

### Capsule Shell Percentage

The percentage of the shell in the capsule (shell percentage) was calculated as follows: shell percentage (%)=capsule shell weight/total capsule weight×100.

The weight was measured using an electronic balance GX-200 manufactured by A&D Company, Limited.

### Capsule Shell Thickness

The thickness of the capsule shells (shell thickness) was measured using a digital microscope manufactured by Keyence Corporation (trade name: VHX-900, using a 10 μm calibration scale),

### Capsule Disintegrability

The produced capsules were tested according to the disintegration test method specified in the Japanese Pharmacopoeia (17th revision). The capsules and the auxiliary disks were placed in a test apparatus, and water at 37±2° C. was added as the test medium. The disintegration time was defined as the time taken for the capsule to clearly lose its original shape or to broke apart with only shell pieces left behind. The number of capsules in the test was at least six per formula, and the average disintegration time of the tested capsules was used as the disintegration time.

### Water Content

The water content was determined by measuring the loss on drying as represented by the following formula.

Loss on drying (%)=[capsule weight (mg)−dry capsule weight (mg)]/capsule weight (mg)×100

The dry capsule weight was defined as the weight of the capsule after it had been thoroughly dried (at 110° C. for 2 hours).

Presence of Cracks after Drying

The capsules that had no cracks after drying were evaluated as “none (0%)”, and those that had cracks were evaluated in terms of the percentage of cracks.

### Examples 1 to 8

A shell-forming liquid was prepared according to the following formula (Table 1), and capsules (contents: MCT (Coconad ML, manufactured by Kao Corporation)) were produced by a drop method using a multi-nozzle under the conditions shown in Table 4.

The gellan gum used was a deacylated type of gellan gum (KELCOGEL, manufactured by CP Kelco). The same applies hereinafter.

The amount of the monovalent metal (sodium) ion in the sodium alginate was 0.0923 g per gram of the sodium alginate. The same applies hereinafter.

TABLE 1

| | | Amount | Amount |
|---|---|---|---|
| | | (% by mass) | (parts by mass) |
| Composition | | relative to | per 100 parts |
| of shell- | Amount | total solids | by mass of |
| forming liquid | (g) | content | gellan gum |
| Gellan gum | 3 | 37.5 | — |
| Sodium alginate | 1 | 12.5 | 33.3 |
| | | (1.15 in terms | (3.08 in terms |
| | | of mass of | of mass of |
| | | monovalent metal) | monovalent metal) |
| Hydrolyzed | 2 | 25 | — |
| guar gum | | | |
| Glycerin | 2 | 25 | — |
| Water | 100 | — | — |

### Comparative Example 1

A shell-forming liquid was prepared according to the following formula (Table 2), and capsules (contents: MCT (Coconad ML, manufactured by Kao Corporation)) were produced by a drop method using a multi-nozzle under the conditions shown in Table 4.

TABLE 2

| | | Amount | Amount |
|---|---|---|---|
| | | (% by mass) | (parts by mass) |
| Composition | | relative to | per 100 parts |
| of shell- | Amount | total solids | by mass of |
| forming liquid | (g) | content | gellan gum |
| Gellan gum | 1.5 | 93.75 | — |
| Calcium lactate | 0.1 | 6.25 | 6.67 |
| | | (0 in terms | (0 in terms |
| | | of mass of | of mass of |
| | | monovalent metal) | monovalent metal) |
| Water | 100 | — | — |

### Examples 9 and 10

A shell-forming liquid was prepared according to the following formula (Table 3), and capsules (contents: MCT (Coconad ML, manufactured by Kao Corporation)) were produced by a drop method using a multi-nozzle under the conditions shown in Table 4. In Example 10, immersion treatment with an aqueous calcium lactate solution was performed before drying.

The amount of the monovalent metal (sodium) ion in the sodium citrate was 0.0891 g per gram of the sodium citrate. The same applies hereinafter.

TABLE 3

| | | Amount | Amount |
|---|---|---|---|
| | | (% by mass) | (parts by mass) |
| Composition | | relative to | per 100 parts |
| of shell- | Amount | total solids | by mass of |
| forming liquid | (g) | content | gellan gum |
| Gellan gum | 2 | 13.5 | — |
| Sorbitol | 1 | 6.76 | — |
| Dextrin | 11.5 | 77.7 | — |
| Sodium citrate | 0.2 | 1.35 | 10.0 |
| | | (0.120 in terms | (0.891 in terms |
| | | of mass of | of mass of |
| | | monovalent metal) | monovalent metal) |
| Citric acid | 0.1 | 0.676 | — |
| Water | 85 | — | — |

The results are shown in Table 4.

TABLE 4

| | Amount | | | | | | | | | | | | | | | | |
|---|---|---|---|---|---|---|---|---|---|---|---|---|---|---|---|---|---|
| | of | | | | | | | | | | | | | | | | |
| | mono- | | | | | | | | | | | | | | | | |
| | valent | | | | | | | | | | | | | | | | |
| | metal | | | | | | | | | | | | | | | | |
| | ion | | | | | | | | | | | | | | | | |
| | (parts | | | | | | | | | | | | | | | | |
| | by | | | | | | | | | | | | | | | | |
| | mass) | | | | | | | | | | | | Crush | | | Crush | |
| | per 100 | Before drying | | | | | | Shell | | | | Crush | stren- | | Crush | deform- | Dis- |
| | parts by | Outer | Water | Crush | Crush | | Outer | per- | Shell | Cap- | Crush | stren- | gth/ | Crush | deform- | ation/ | inte- |
| | mass of | dia- | con- | stren- | strength/ | Cracks | dia- | cen- | thick- | sule | stren- | gth | Outer | deform- | ation | Outer | gration |
| | gellan | meter | tent | gth | Outer | after | meter | tage | ness | weight | gth | SD | dia- | ation | SD | dia- | time |
| | gum | (mm) | (%) | (g) | diameter | drying | (mm) | (%) | (μm) | (mg) | (g) | (g) | meter | (mm) | (mm) | meter | (min) |
| Ex- | 3.08 | 5.2 | 92.6 | 40 | 7.7 | None | 4 | 10 | 62 | 31.9 | 1000 | 240 | 250 | 1.8 | 0.2 | 0.45 | 5 min |
| ample | | | | | | | | | | | | | | | | | |
| 1 | | | | | | | | | | | | | | | | | |
| Ex- | 3.08 | 5.7 | 92.6 | 50 | 8.8 | None | 4 | 15 | 95 | 32.2 | 1300 | 260 | 325 | 2.0 | 0.2 | 0.50 | 10 min |
| ample | | | | | | | | | | | | | | | | | |
| 2 | | | | | | | | | | | | | | | | | |
| Ex- | 3.08 | 7.5 | 92.6 | 60 | 8.0 | None | 6 | 8 | 72 | 107.2 | 2000 | 280 | 333 | 2.4 | 0.3 | 0.40 | 5 min |
| ample | | | | | | | | | | | | | | | | | |
| 3 | | | | | | | | | | | | | | | | | |
| Ex- | 3.08 | 7.9 | 92.6 | 70 | 8.9 | None | 6 | 12 | 142 | 108.0 | 2400 | 300 | 400 | 2.5 | 0.3 | 0.42 | 10 min |
| ample | | | | | | | | | | | | | | | | | |
| 4 | | | | | | | | | | | | | | | | | |
| Ex- | 3.08 | 9.2 | 92.6 | 80 | 8.5 | None | 8 | 5 | 54 | 253 | 2800 | 300 | 325 | 2.6 | 0.3 | 0.33 | 5 min |
| ample | | | | | | | | | | | | | | | | | |
| 5 | | | | | | | | | | | | | | | | | |
| Ex- | 3.08 | 9.7 | 92.6 | 90 | 9.3 | None | 8 | 7 | 84 | 254 | 2800 | 300 | 350 | 2.7 | 0.3 | 0.34 | 5 min |
| ample | | | | | | | | | | | | | | | | | |
| 6 | | | | | | | | | | | | | | | | | |
| Ex- | 3.08 | 9.9 | 92.6 | 100 | 10.1 | None | 8 | 8 | 94 | 254 | 3000 | 320 | 375 | 2.8 | 0.3 | 0.35 | 5 min |
| ample | | | | | | | | | | | | | | | | | |
| 7 | | | | | | | | | | | | | | | | | |
| Ex- | 3.08 | 10.8 | 92.6 | 140 | 13.0 | None | 8 | 12 | 150 | 256 | 3600 | 340 | 450 | 3.2 | 0.4 | 0.40 | 10 min |
| ample | | | | | | | | | | | | | | | | | |
| 8 | | | | | | | | | | | | | | | | | |
| Com- | 0.00 | 7.1 | 98.4 | 30 | 4.2 | 75% | 4 | 8 | 95 | 31.7 | 800 | 270 | 200 | 1.4 | 0.3 | 0.35 | >60 min |
| para- | | | | | | | | | | | | | | | | | |
| tive | | | | | | | | | | | | | | | | | |
| Ex- | | | | | | | | | | | | | | | | | |
| ample | | | | | | | | | | | | | | | | | |
| 1 | | | | | | | | | | | | | | | | | |
| Ex- | 0.891 | 4.5 | 85.2 | 20 | 4.4 | 25% | 4 | 8 | 95 | 31.7 | 1000 | 280 | 250 | 1.6 | 0.2 | 0.40 | 10 min |
| ample | | | | | | | | | | | | | | | | | |
| 9 | | | | | | | | | | | | | | | | | |
| Ex- | 0.891 | 4.5 | 85.2 | 40 | 8.9 | None | 4 | 8 | 95 | 31.7 | 2500 | 340 | 625 | 1.8 | 0.3 | 0.45 | >60 min |
| ample | | | | | | | | | | | | | | | | | |
| 10 | | | | | | | | | | | | | | | | | |

As is evident from the results in the above table, capsules having excellent water disintegrability and excellent mechanical strength were obtained in the examples.

In particular, comparison between the results of Example 9 and others shows that appropriate selection of the capsule production conditions (the conditions before drying) makes it possible to efficiently produce crack-free capsules after drying.

The results of Example 10 show that the immersion treatment before drying increases the mechanical strength of capsules but reduces the water disintegrability of capsules.

In contrast, in Examples 1 to 8, crack-free capsules having both water disintegrability and excellent mechanical strength were obtained.

### Examples 11 to 19

A shell-forming liquid was prepared according to the following formulae (Table 5), and capsules (contents: MCT (Coconad ML, manufactured by Kao Corporation)) were produced by a drop method using a multi-nozzle under the conditions shown in Table 6.

TABLE 5

| Composition of | Amount (g) | | | | |
|---|---|---|---|---|---|
| shell-forming | Examples | Examples | Examples | Example | Examples |
| liquid | 11&12 | 13&14 | 15&16 | 17 | 18&19 |
| Gellan gum | 1.5 | 2.5 | 2.5 | 2.5 | 1 |
| Agar | 1.5 | 0.5 | — | — | 3 |
| Carrageenan | — | — | 0.5 | — | — |
| Pectin | — | — | — | 0.5 | 14+ |
| Sodium alginate | 1 | 1 | 1 | 1 | 1 |
| Hydrolyzed | 2 | 2 | 2 | 2 | 2 |
| guar gum | | | | | |
| Glycerin | 2 | 2 | 2 | 2 | 2 |
| Water | 100 | 100 | 100 | 100 | 100 |

In the shell-forming liquid, the amount of the gellan gum relative to the total solids content was 18.75% by mass (Examples 11 and 12), 31.25% by mass (Examples 13 to 17), and 12.5% by mass (Examples 18 and 19); the amount of the sodium alginate relative to the total solids content was 12.5% by mass (0.092% by mass in terms of the mass of the monovalent metal); and the amount of the sodium alginate relative to 100 parts by mass of the gellan gum was 66.7 parts by mass (6.15 parts by mass in terms of the mass of the monovalent metal) [Examples 11 and 12], 40.0 parts by mass (3.69 parts by mass in terms of the mass of the monovalent metal) [Examples 13 to 17], and 100 parts by mass (9.23 parts by mass in terms of the mass of the monovalent metal) [Examples 18 and 19].

The results are shown in Table 6.

TABLE 6

| | Amount | | | | | | | | | | | | | | | | |
|---|---|---|---|---|---|---|---|---|---|---|---|---|---|---|---|---|---|
| | of | | | | | | | | | | | | | | | | |
| | mono- | | | | | | | | | | | | | | | | |
| | valent | | | | | | | | | | | | | | | | |
| | metal | | | | | | | | | | | | | | | | |
| | ion | | | | | | | | | | | | | | | | |
| | (parts | | | | | | | | | | | | | | | | |
| | by | | | | | | | | | | | | | | | | |
| | mass) | Before drying | | | | | | | | | | | Crush | | | Crush | |
| | per 100 | | | | Crush | | | Shell | | Cap- | | Crush | stren- | | Crush | deform- | Dis- |
| | parts by | Outer | Water | Crush | strength/ | | Outer | per- | Shell | sule | Crush | stren- | gth/ | Crush | deform- | ation/ | inte- |
| | mass of | dia- | con- | stren- | Outer | Cracks | dia- | cen- | thick- | wei- | stren- | gth | Outer | deform- | ation | Outer | gration |
| | gellan | meter | tent | gth | dia- | after | meter | tage | ness | ght | gth | SD | dia- | ation | SD | dia- | time |
| | gum | (mm) | (%) | (g) | meter | drying | (mm) | (%) | (μm) | (mg) | (g) | (g) | meter | (mm) | (mm) | meter | (min) |
| Ex- | 6.15 | 5 | 92.6 | 30 | 6.0 | None | 4 | 8 | 96 | 31.7 | 1200 | 230 | 300 | 2.0 | 0.3 | 0.50 | 5 min |
| ample | | | | | | | | | | | | | | | | | |
| 11 | | | | | | | | | | | | | | | | | |
| Ex- | 6.15 | 9.9 | 92.6 | 100 | 10.1 | None | 8 | 8 | 94 | 254 | 3600 | 370 | 450 | 2.9 | 0.4 | 0.36 | 10 min |
| ample | | | | | | | | | | | | | | | | | |
| 12 | | | | | | | | | | | | | | | | | |
| Ex- | 3.69 | 7.5 | 92.6 | 60 | 8.0 | None | 6 | 8 | 72 | 107 | 1700 | 290 | 283 | 2.5 | 0.3 | 0.42 | 5 min |
| ample | | | | | | | | | | | | | | | | | |
| 13 | | | | | | | | | | | | | | | | | |
| Ex- | 3.69 | 9.5 | 92.6 | 90 | 9.2 | None | 8 | 8 | 94 | 254 | 3200 | 330 | 400 | 2.8 | 0.3 | 0.35 | 5 min |
| ample | | | | | | | | | | | | | | | | | |
| 14 | | | | | | | | | | | | | | | | | |
| Ex- | 3.89 | 7.5 | 92.6 | 60 | 8.0 | None | 6 | 8 | 72 | 107 | 1600 | 270 | 267 | 2.5 | 0.3 | 0.42 | 5 min |
| ample | | | | | | | | | | | | | | | | | |
| 15 | | | | | | | | | | | | | | | | | |
| Ex- | 3.69 | 9.8 | 92.6 | 90 | 9.2 | None | 8 | 8 | 94 | 254 | 3100 | 310 | 388 | 2.8 | 0.3 | 0.35 | 5 min |
| ample | | | | | | | | | | | | | | | | | |
| 16 | | | | | | | | | | | | | | | | | |
| Ex- | 3.69 | 7.5 | 92.6 | 60 | 6.7 | None | 6 | 8 | 72 | 107 | 1500 | 250 | 250 | 2.3 | 0.3 | 0.38 | 5 min |
| ample | | | | | | | | | | | | | | | | | |
| 17 | | | | | | | | | | | | | | | | | |
| Ex- | 9.23 | 9.2 | 92.6 | 40 | 4.3 | 20% | 8 | 5 | 54 | 253 | 3200 | 390 | 400 | 2.7 | 0.5 | 0.34 | >60 min |
| ample | | | | | | | | | | | | | | | | | |
| 18 | | | | | | | | | | | | | | | | | |
| Ex- | 9.23 | 9.8 | 92.6 | 90 | 9.2 | None | 8 | 8 | 94 | 254 | 4200 | 470 | 525 | 3.0 | 0.4 | 0.38 | >60 min |
| ample | | | | | | | | | | | | | | | | | |
| 19 | | | | | | | | | | | | | | | | | |

The above table shows that capsules having sufficient water disintegrability and sufficient mechanical strength can be obtained even when gellan gum is combined with another shell-forming base material (agar, carrageenan, or pectin).

In particular, comparison between the results of Examples 11 to 17 and the results of Examples 18 and 19 shows that appropriate selection of the amount (not too small an amount) of the gellan gum relative to the total amount of the shell-forming base materials makes it possible to efficiently achieve both water disintegrability and mechanical strength.

Comparison between the results of Example 18 and others shows that even when another shell-forming base material is used in combination with gellan gum, appropriate selection of the capsule production conditions (the conditions before drying) tends to make it possible to efficiently produce crack-free capsules after drying.

### Examples 20 to 26

A shell-forming liquid was prepared according to the following formulae (Table 7), and capsules (contents: MCT (Coconad ML, manufactured by Kao Corporation)) were produced by a drop method using a multi-nozzle under the conditions shown in Table 8. For comparison, Example 7 is also shown.

The amount of the monovalent metal (potassium) ion in the potassium alginate was 0.112 g per gram of the potassium alginate. The same applies hereinafter.

TABLE 7

| | Amount (g) | | | | | | | |
|---|---|---|---|---|---|---|---|---|
| Composition of | Example | Example | Example | Example | Example | Example | Example | Example |
| shell-forming liquid | 7 | 20 | 21 | 22 | 23 | 24 | 25 | 26 |
| Gellan gum | 3 | 3 | 3 | 3 | 3 | 3 | 3 | 3 |
| NaCl | — | 0.3 | — | 0.2 | — | — | — | — |
| KCl | — | — | 0.3 | — | 0.2 | — | — | — |
| Sodium alginate | 1 | — | — | 1 | 1 | 3 | 1 | — |
| Potassium alginate | — | — | — | — | — | — | 2 | 3 |
| Hydrolyzed guar gum | 2 | 2.7 | 2.7 | 1.8 | 1.8 | — | — | — |
| Glycerin | 2 | 2 | 2 | 2 | 2 | 2 | 2 | 2 |
| Water | 100 | 100 | 100 | 100 | 100 | 100 | 100 | 100 |

In the shell-forming liquid, the amount of the gellan gum relative to the total solids content was 37.5% by mass; the amount of the monovalent metal compound relative to the total solids content was 3.75% by mass (1.48% by mass in terms of the mass of the monovalent metal) [Example 20], 3.75% by mass (1.97% by mass in terms of the mass of the monovalent metal) [Example 21], 15.0% by mass (2.14% by mass in terms of the mass of the monovalent metal) [Example 22], 15.0% by mass (2.47% by mass in terms of the mass of the monovalent metal) [Example 23], 37.5% by mass (3.46% by mass in terms of the mass of the monovalent metal) [Example 24], 37.5% by mass (3.95% by mass in terms of the mass of the monovalent metal) [Example 25], and 37.5% by mass (4.20% by mass in terms of the mass of the monovalent metal) [Example 26]; and the amount of the monovalent metal compound relative to 100 parts by mass of the gellan gum was 10.0 parts by mass (3. 93 parts by mass in terms of the mass of the monovalent metal) [Example 20], 10.0 parts by mass (5.25 parts by mass in terms of the mass of the monovalent metal) [Example 21], 40.0 parts by mass (5.70 parts by mass in terms of the mass of the monovalent metal) [Example 22], 40.0 parts by mass (6.57 parts by mass in terms of the mass of the monovalent metal) [Example 23], 100 parts by mass (9.23 parts by mass in terms of the mass of the monovalent metal) [Example 24], 100 parts by mass (10.54 parts by mass in terms of the mass of the monovalent metal) [Example 25], and 100 parts by mass (11.20 parts by mass in terms of the mass of the monovalent metal) [Example 26].

The results are shown in Table 8.

TABLE 8

| | Amount | | | | | | | | | | | | | | | | |
|---|---|---|---|---|---|---|---|---|---|---|---|---|---|---|---|---|---|
| | of | | | | | | | | | | | | | | | | |
| | mono- | | | | | | | | | | | | | | | | |
| | valent | | | | | | | | | | | | | | | | |
| | metal | | | | | | | | | | | | | | | | |
| | ion | | | | | | | | | | | | | | | | |
| | (parts | | | | | | | | | | | | | | | | |
| | by | | | | | | | | | | | | | | | | |
| | mass) | | | | | | | | | | | | Crush | | | Crush | |
| | per 100 | Before drying | | | | | | Shell | | | | Crush | stren- | | Crush | deform- | Dis- |
| | parts by | Outer | Water | Crush | Crush | | Outer | per- | Shell | Cap- | Crush | stren- | gth/ | Crush | deform- | ation/ | inte- |
| | mass of | dia- | con- | stren- | strength/ | Cracks | dia- | cen- | thick- | sule | stren- | gth | Outer | deform- | ation | Outer | gration |
| | gellan | meter | tent | gth | Outer | after | meter | tage | ness | weight | gth | SD | dia- | ation | SD | dia- | time |
| | gum | (mm) | (%) | (g) | diameter | drying | (mm) | (%) | (μm) | (mg) | (g) | (g) | meter | (mm) | (mm) | meter | (min) |
| Ex- | 3.08 | 9.9 | 92.6 | 100 | 10.1 | None | 8 | 8 | 94 | 254 | 3000 | 320 | 376 | 2.8 | 0.3 | 0.35 | 5 min |
| ample | | | | | | | | | | | | | | | | | |
| 7 | | | | | | | | | | | | | | | | | |
| Ex- | 3.93 | 9.9 | 92.6 | 110 | 11.1 | None | 8 | 8 | 94 | 254 | 3200 | 360 | 400 | 2.8 | 0.4 | 0.35 | 10 min |
| ample | | | | | | | | | | | | | | | | | |
| 20 | | | | | | | | | | | | | | | | | |
| Ex- | 5.25 | 9.9 | 92.6 | 100 | 10.1 | None | 8 | 8 | 94 | 254 | 2800 | 380 | 350 | 2.8 | 0.5 | 0.35 | 10 min |
| ample | | | | | | | | | | | | | | | | | |
| 21 | | | | | | | | | | | | | | | | | |
| Ex- | 5.70 | 9.9 | 92.6 | 140 | 14.1 | None | 8 | 8 | 94 | 254 | 4000 | 390 | 500 | 3.0 | 0.4 | 0.38 | 5 min |
| ample | | | | | | | | | | | | | | | | | |
| 22 | | | | | | | | | | | | | | | | | |
| Ex- | 6.57 | 9.9 | 92.6 | 120 | 12.1 | None | 8 | 8 | 94 | 254 | 3500 | 420 | 438 | 3.0 | 0.5 | 0.38 | 5 min |
| ample | | | | | | | | | | | | | | | | | |
| 23 | | | | | | | | | | | | | | | | | |
| Ex- | 9.23 | 9.9 | 92.6 | 160 | 16.2 | None | 8 | 8 | 94 | 254 | 4200 | 300 | 525 | 3.3 | 0.2 | 0.41 | 5 min |
| ample | | | | | | | | | | | | | | | | | |
| 24 | | | | | | | | | | | | | | | | | |
| Ex- | 10.54 | 9.9 | 92.6 | 150 | 15.2 | None | 8 | 8 | 94 | 254 | 4000 | 400 | 500 | 2.9 | 0.3 | 0.36 | 5 min |
| ample | | | | | | | | | | | | | | | | | |
| 25 | | | | | | | | | | | | | | | | | |
| Ex- | 11.20 | 9.9 | 92.6 | 140 | 14.1 | None | 8 | 8 | 94 | 254 | 3800 | 440 | 475 | 2.8 | 0.4 | 0.35 | 10 min |
| ample | | | | | | | | | | | | | | | | | |
| 26 | | | | | | | | | | | | | | | | | |

As is evident from the results in the above tale, capsules having excellent water disintegrability and excellent mechanical strength were obtained even though various monovalent alkali metal salts were used.

### Examples 27 to 31

A shell-forming liquid was prepared according to the following formulae (Table 9), and capsules (contents: MCT (Coconad ML, manufactured by Kao Corporation)) were produced by a drop method using a multi-nozzle under the conditions shown in Table 10. For comparison, Example 24 is also shown.

TABLE 9

| Composition of | Amount (g) | | | | | |
|---|---|---|---|---|---|---|
| shell-forming | Example | Example | Example | Example | Example | Example |
| liquid | 24 | 27 | 28 | 29 | 30 | 31 |
| Gellan gum | 3 | 3 | 3 | 3 | 3 | 3 |
| Sodium alginate | 3 | 3 | 3 | 3 | 3 | 3 |
| Glycerin | 2 | 4 | 2 | 2 | 2 | 2 |
| Propylene glycol | — | — | 2 | — | — | — |
| Sorbitol | — | — | — | 2 | — | — |
| Erythritol | — | — | — | — | 2 | — |
| Trehalose | — | — | — | — | — | 2 |
| Water | 100 | 100 | 100 | 100 | 100 | 100 |

In the shell-forming liquid, the amount of the gellan gum relative to the total solids content was 30.0% by mass; the amount of the sodium alginate relative to the total solids content was 30.0% by mass (2.77% by mass in terms of the mass of the monovalent metal) ; and the amount of the sodium alginate relative to 100 parts by mass of the gellan gum was 100 parts by mass (9. 23 parts by mass in terms of the mass of the monovalent metal).

The results are shown in Table 10.

TABLE 10

| | Amount | | | | | | | | | | | | | | | | |
|---|---|---|---|---|---|---|---|---|---|---|---|---|---|---|---|---|---|
| | of | | | | | | | | | | | | | | | | |
| | mono- | | | | | | | | | | | | | | | | |
| | valent | | | | | | | | | | | | | | | | |
| | metal | | | | | | | | | | | | | | | | |
| | ion | | | | | | | | | | | | | | | | |
| | (parts | | | | | | | | | | | | | | | | |
| | by | | | | | | | | | | | | | | | | |
| | mass) | | | | | | | | | | | | Crush | | | Crush | |
| | per 100 | Before drying | | | | | | Shell | | | | Crush | stren- | | Crush | deform- | Dis- |
| | parts by | Outer | Water | Crush | Crush | | Outer | per- | Shell | Cap- | Crush | stren- | gth/ | Crush | deform- | ation/ | inte- |
| | mass of | dia- | con- | stren- | strength/ | Cracks | dia- | cen- | thick- | sule | stren- | gth | Outer | deform- | ation | Outer | gration |
| | gellan | meter | tent | gth | Outer | after | meter | tage | ness | weight | gth | SD | dia- | ation | SD | dia- | time |
| | gum | (mm) | (%) | (g) | diameter | drying | (mm) | (%) | (μm) | (mg) | (g) | (g) | meter | (mm) | (mm) | meter | (min) |
| Ex- | 9.23 | 9.9 | 92.6 | 160 | 16.2 | None | 8 | 8 | 94 | 254 | 4200 | 300 | 525 | 3.3 | 0.2 | 0.41 | 5 min |
| ample | | | | | | | | | | | | | | | | | |
| 24 | | | | | | | | | | | | | | | | | |
| Ex- | 9.23 | 9.7 | 90.9 | 150 | 15.5 | None | 8 | 8 | 94 | 254 | 4900 | 320 | 613 | 3.6 | 0.3 | 0.45 | 5 min |
| ample | | | | | | | | | | | | | | | | | |
| 27 | | | | | | | | | | | | | | | | | |
| Ex- | 9.23 | 97 | 90.9 | 140 | 14.4 | None | 8 | 8 | 94 | 254 | 4600 | 320 | 575 | 3.5 | 0.3 | 0.44 | 5 min |
| ample | | | | | | | | | | | | | | | | | |
| 28 | | | | | | | | | | | | | | | | | |
| Ex- | 9.23 | 9.7 | 90.9 | 150 | 15.5 | None | 8 | 8 | 94 | 254 | 4400 | 310 | 550 | 3.4 | 0.3 | 0.43 | 5 min |
| ample | | | | | | | | | | | | | | | | | |
| 29 | | | | | | | | | | | | | | | | | |
| Ex- | 9.23 | 9.7 | 90.9 | 150 | 15.5 | None | 8 | 8 | 94 | 254 | 4800 | 270 | 600 | 3.6 | 0.3 | 0.45 | 5 min |
| ample | | | | | | | | | | | | | | | | | |
| 30 | | | | | | | | | | | | | | | | | |
| Ex- | 9.23 | 9.8 | 90.9 | 150 | 15.3 | None | 8 | 8 | 94 | 254 | 4800 | 290 | 600 | 3.6 | 0.3 | 0.45 | 5 min |
| ample | | | | | | | | | | | | | | | | | |
| 31 | | | | | | | | | | | | | | | | | |

As is evident from the results in the above table, capsules having excellent water disintegrability and excellent mechanical strength were obtained even though various plasticizers were used.

### Examples 32 and 33

A shell-forming liquid was prepared according to the following formula (Table 11), and capsules consisting only of a shell without contents (monolayer spheres) were produced by a drop method using a single nozzle.

TABLE 11

| Composition | |
|---|---|
| of shell- | |
| forming liquid | Amount (g) |
| Gellan gum | 3 |
| Sodium alginate | 4 |
| Hydrolyzed guar gum | 4 |
| Glycerin | 4 |
| Water | 70 |

In the shell-forming liquid, the amount of the gellan gum relative to the total solids content was 20.0% by mass; the amount of the sodium alginate relative to the total solids content was 26.7% by mass; and the amount of the sodium alginate relative to 100 parts by mass of the gellan gum was 133 parts by mass (12.31 parts by mass in terms of the mass of the monovalent metal).

The results are shown in Table 12.

TABLE 12

| | Amount | | | | | | | | | | | | | | | | |
|---|---|---|---|---|---|---|---|---|---|---|---|---|---|---|---|---|---|
| | of | | | | | | | | | | | | | | | | |
| | metal | | | | | | | | | | | | | | | | |
| | ion | | | | | | | | | | | | | | | | |
| | (parts | | | | | | | | | | | | | | | | |
| | by | | | | | | | | | | | | | | | | |
| | mass) | | | | | | | | | | | | Crush | | | Crush | |
| | per 100 | Before drying | | | | | | Shell | | | | Crush | stren- | | Crush | deform- | Dis- |
| | parts by | Outer | Water | Crush | Crush | | Outer | per- | Shell | Cap- | Crush | stren- | gth/ | Crush | deform- | ation/ | inte- |
| | mass of | dia- | con- | stren- | strength/ | Cracks | dia- | cen- | thick- | sule | stren- | gth | Outer | deform- | ation | Outer | gration |
| | gellan | meter | tent | gth | Outer | after | meter | tage | ness | weight | gth | SD | dia- | ation | SD | dia- | time |
| | gum | (mm) | (%) | (g) | diameter | drying | (mm) | (%) | (μm) | (mg) | (g) | (g) | meter | (mm) | (mm) | meter | (min) |
| Ex- | 12.31 | 5.4 | 82.4 | 1000 | 185 | None | 3 | 100 | 3000 | 15.0 | 12000 | 800 | 4000 | 2.8 | 0.2 | 0.93 | 30 min |
| ample | | | | | | | | | | | | | | | | | |
| 32 | | | | | | | | | | | | | | | | | |
| Ex- | 12.31 | 9.1 | 82.4 | 1800 | 198 | None | 5 | 100 | 5000 | 69.5 | 16000 | 1400 | 3200 | 4.7 | 0.2 | 0.94 | 40 min |
| ample | | | | | | | | | | | | | | | | | |
| 33 | | | | | | | | | | | | | | | | | |

As is evident from the results in the above table, even without contents, capsules having excellent water disintegrability and excellent mechanical strength were obtained.

### Examples 34 and 35

A shell-forming liquid was prepared according to the same formula as in Example 24 (Table 9), and capsules (contents of Example 34: refined olive oil (Olive Oil Refined, manufactured by Dcoop S. Coop. And.), contents of Example 35: 80% by mass fish oil (DHA-46MK, manufactured by Maruha Nichiro Corporation) and 20% by mass MCT (Coconad ML, manufactured by Kao Corporation)) were produced by a drop method using a multi-nozzle under the conditions shown in Table 13. For comparison, Example 24 is also shown,

The results are shown in Table 13.

TABLE 13

| | Amount | | | | | | | | | | | | | | | | |
|---|---|---|---|---|---|---|---|---|---|---|---|---|---|---|---|---|---|
| | of | | | | | | | | | | | | | | | | |
| | mono- | | | | | | | | | | | | | | | | |
| | valent | | | | | | | | | | | | | | | | |
| | metal | | | | | | | | | | | | | | | | |
| | ion | | | | | | | | | | | | | | | | |
| | (parts | | | | | | | | | | | | | | | | |
| | by | | | | | | | | | | | | | | | | |
| | mass) | | | | | | | | | | | | Crush | | | Crush | |
| | per 100 | Before drying | | | | | | Shell | | | | Crush | stren- | | Crush | deform- | Dis- |
| | parts by | Outer | Water | Crush | Crush | | Outer | per- | Shell | Cap- | Crush | stren- | gth/ | Crush | deform- | ation/ | inte- |
| | mass of | dia- | con- | stren- | strength/ | Cracks | dia- | cen- | thick- | sule | stren- | gth | Outer | deform- | ation | Outer | gration |
| | gellan | meter | tent | gth | Outer | after | meter | tage | ness | weight | gth | SD | dia- | ation | SD | dia- | time |
| | gum | (mm) | (%) | (g) | diameter | drying | (mm) | (%) | (μm) | (mg) | (g) | (g) | meter | (mm) | (mm) | meter | (min) |
| Ex- | 9.23 | 9.9 | 92.6 | 160 | 16.2 | None | 8 | 8 | 94 | 254 | 4200 | 300 | 525 | 3.3 | 0.2 | 0.41 | 5 min |
| ample | | | | | | | | | | | | | | | | | |
| 24 | | | | | | | | | | | | | | | | | |
| Ex- | 9.23 | 9.9 | 92.6 | 160 | 16.2 | None | 8 | 8 | 93 | 248 | 4200 | 300 | 525 | 3.3 | 0.2 | 0.41 | 5 min |
| ample | | | | | | | | | | | | | | | | | |
| 34 | | | | | | | | | | | | | | | | | |
| Ex- | 9.23 | 9.9 | 92.8 | 150 | 15.2 | None | 8 | 8 | 94 | 256 | 4000 | 320 | 500 | 3.2 | 0.3 | 0.40 | 5 min |
| ample | | | | | | | | | | | | | | | | | |
| 35 | | | | | | | | | | | | | | | | | |

As is evident from the results in the above table, capsules having excellent water disintegrability and excellent mechanical strength were obtained even though the contents were varied.

### Examples 36 to 43

A shell-forming liquid was prepared according to the following formula (Table 14), which did not contain sodium alginate and contained hydrolyzed guar gum in an amount corresponding to the combined total amount of sodium alginate and hydrolyzed guar gum shown in Table 1 (Examples 1 to 8). Capsules (contents: MCT (Coconad ML, manufactured by Kao Corporation)) were produced by a drop method using a multi-nozzle under the conditions shown in Table 15. For Comparison, Examples 1 to 8 are also shown.

TABLE 14

| | | Amount |
|---|---|---|
| | | (% by mass) |
| Composition | | relative to |
| of shell- | Amount | total solids |
| forming liquid | (g) | content |
| Gellan gum | 3 | 37.5 |
| Hydrolyzed guar gum | 3 | 37.5 |
| Glycerin | 2 | 25.0 |
| Water | 100 | — |

The results are shown in Table 15.

TABLE 15

| | Amount | | | | | | | | | | | | | | | | |
|---|---|---|---|---|---|---|---|---|---|---|---|---|---|---|---|---|---|
| | of | | | | | | | | | | | | | | | | |
| | mono- | | | | | | | | | | | | | | | | |
| | valent | | | | | | | | | | | | | | | | |
| | metal | | | | | | | | | | | | | | | | |
| | ion | | | | | | | | | | | | | | | | |
| | (parts | | | | | | | | | | | | | | | | |
| | by | | | | | | | | | | | | | | | | |
| | mass) | | | | | | | | | | | | Crush | | | Crush | |
| | per 100 | Before drying | | | | | | Shell | | | | Crush | stren- | | Crush | deform- | Dis- |
| | parts by | Outer | Water | Crush | Crush | | Outer | per- | Shell | Cap- | Crush | stren- | gth/ | Crush | deform- | ation/ | inte- |
| | mass of | dia- | con- | stren- | strength/ | Cracks | dia- | cen- | thick- | sule | stren- | gth | Outer | deform- | ation | Outer | gration |
| | gellan | meter | tent | gth | Outer | after | meter | tage | ness | weight | gth | SD | dia- | ation | SD | dia- | time |
| | gum | (mm) | (%) | (g) | diameter | drying | (mm) | (%) | (μm) | (mg) | (g) | (g) | meter | (mm) | (mm) | meter | (min) |
| Ex- | 3.08 | 5.2 | 92.6 | 40 | 7.7 | None | 4 | 10 | 62 | 31.9 | 1000 | 240 | 250 | 1.8 | 0.2 | 0.45 | 5 min |
| ample | | | | | | | | | | | | | | | | | |
| 1 | | | | | | | | | | | | | | | | | |
| Ex- | — | 5.2 | 92.6 | 25 | 4.8 | 25% | 4 | 10 | 62 | 31.9 | 500 | 160 | 125 | 1.2 | 0.2 | 0.3 | 20 min |
| ample | | | | | | | | | | | | | | | | | |
| 36 | | | | | | | | | | | | | | | | | |
| Ex- | 3.08 | 5.7 | 92.6 | 50 | 8.8 | None | 4 | 15 | 95 | 32.2 | 1300 | 260 | 325 | 2.0 | 0.2 | 0.60 | 10 min |
| ample | | | | | | | | | | | | | | | | | |
| 2 | | | | | | | | | | | | | | | | | |
| Ex- | — | 5.7 | 92.8 | 30 | 5.3 | 15% | 4 | 15 | 95 | 32.2 | 800 | 220 | 200 | 1.7 | 0.3 | 0.43 | 40 min |
| ample | | | | | | | | | | | | | | | | | |
| 37 | | | | | | | | | | | | | | | | | |
| Ex- | 3.08 | 7.5 | 92.8 | 60 | 8.0 | None | 6 | 8 | 72 | 107.2 | 2000 | 280 | 333 | 2.4 | 0.3 | 0.40 | 5 min |
| ample | | | | | | | | | | | | | | | | | |
| 3 | | | | | | | | | | | | | | | | | |
| Ex- | | 7.5 | 92.6 | 35 | 4.7 | 30% | 6 | 8 | 72 | 107.2 | 1200 | 260 | 200 | 1.9 | 0.4 | 0.32 | 25 min |
| ample | | | | | | | | | | | | | | | | | |
| 38 | | | | | | | | | | | | | | | | | |
| Ex- | 3.08 | 7.9 | 92.6 | 70 | 8.9 | None | 6 | 12 | 142 | 108.0 | 2400 | 300 | 400 | 2.5 | 0.3 | 0.42 | 10 min |
| ample | | | | | | | | | | | | | | | | | |
| 4 | | | | | | | | | | | | | | | | | |
| Ex | — | 7.9 | 92.6 | 40 | 5.1 | 10% | 6 | 12 | 142 | 108 | 1400 | 350 | 230 | 2.1 | 0.4 | 0.35 | 45 min |
| ample | | | | | | | | | | | | | | | | | |
| 39 | | | | | | | | | | | | | | | | | |
| Ex- | 3.08 | 9.2 | 92.6 | 60 | 6.5 | None | 8 | 5 | 54 | 253 | 2600 | 300 | 325 | 2.6 | 0.3 | 0.33 | 5 min |
| ample | | | | | | | | | | | | | | | | | |
| 5 | | | | | | | | | | | | | | | | | |
| Ex- | — | 9.2 | 92.6 | 35 | 3.8 | 70% | 8 | 5 | 54 | 253 | 1600 | 400 | 200 | 2.2 | 0.4 | 0.28 | 10 min |
| ample | | | | | | | | | | | | | | | | | |
| 40 | | | | | | | | | | | | | | | | | |
| Ex- | 3.08 | 9.7 | 92.6 | 90 | 9.3 | None | 8 | 7 | 84 | 254 | 2800 | 300 | 350 | 2.7 | 0.3 | 0.34 | 5 min |
| ample | | | | | | | | | | | | | | | | | |
| 6 | | | | | | | | | | | | | | | | | |
| Ex- | — | 9.7 | 92.6 | 55 | 5.7 | 25% | 8 | 7 | 84 | 254 | 1700 | 400 | 213 | 2.3 | 0.4 | 0.29 | 30 min |
| ample | | | | | | | | | | | | | | | | | |
| 41 | | | | | | | | | | | | | | | | | |
| Ex- | 3.08 | 9.9 | 92.6 | 100 | 10.1 | None | 8 | 8 | 94 | 254 | 3000 | 320 | 375 | 2.8 | 0.3 | 0.35 | 5 min |
| ample | | | | | | | | | | | | | | | | | |
| 2 | | | | | | | | | | | | | | | | | |
| Ex- | — | 9.9 | 92.6 | 60 | 6.0 | 20% | 8 | 8 | 94 | 254 | 1800 | 400 | 225 | 2.3 | 0.4 | 0.29 | 40 min |
| ample | | | | | | | | | | | | | | | | | |
| 42 | | | | | | | | | | | | | | | | | |
| Ex- | 3.08 | 10.8 | 92.6 | 140 | 13.0 | None | 8 | 12 | 150 | 256 | 3600 | 340 | 450 | 3.2 | 0.4 | 0.40 | 10 min |
| ample | | | | | | | | | | | | | | | | | |
| 8 | | | | | | | | | | | | | | | | | |
| Ex- | — | 10.8 | 92.6 | 80 | 704 | 10% | 8 | 12 | 150 | 156 | 2100 | 440 | 263 | 2.4 | 0.4 | 0.30 | 60 min |
| ample | | | | | | | | | | | | | | | | | |
| 43 | | | | | | | | | | | | | | | | | |

### INDUSTRIAL APPLICABILITY

The capsule of the present invention is suitable for use in the fields of medicine, food, industry, etc., for example.

1. A soft capsule having a shell which comprises gellan gum and is substantially free of gelatin, wherein the soft capsule satisfies the following requirements:
(A) the soft capsule has a disintegration time of 60 minutes or less as measured by a disintegration test method specified in the Japanese Pharmacopoeia using water as a test medium; and/or
(B) the soft capsule has a ratio of a crush strength (g) to an outer diameter (mm) (crush strength/outer diameter ratio) of 210 or more.

2. A soft capsule having a shell which comprises gellan gum and a monovalent metal ion and is substantially free of gelatin.

3. The capsule according to claim 1, wherein (A) the capsule has a disintegration time of 60 minutes or less as measured by a disintegration test method specified in the Japanese Pharmacopoeia using water as a test medium; and (B) the capsule has a ratio of a crush strength (g) to an outer diameter (mm) (crush strength/outer diameter ratio) of 210 or more.

4. The capsule according to any one of claims 1 to 3 claim 1, wherein (C) the capsule has a ratio of a crush deformation (mm) to an outer diameter (mm) (crush deformation/outer diameter ratio) of 0.1 or more.

5. The capsule according to claim 1, wherein the amount of the gellan gum in the shell is 5% by mass or more.

6. The capsule according to claim 1, wherein the monovalent metal ion is contained in the form of a monovalent metal compound.

7. The capsule according to claim 1, wherein the monovalent metal ion is contained in the form of at least one monovalent metal compound selected from alkali metal halides, alkali metal salts of organic acids, and alkali metal salts of sugars or polysaccharides.

8. The capsule according to claim 1, wherein the monovalent metal ion is contained at least in the form of an alkali metal salt of alginic acid.

9. The capsule according to claim 1, wherein the amount of the monovalent metal ion is 0.1 part by mass or more in terms of the mass of a metal atom relative to 100 parts by mass of the gellan gum.

10. The capsule according to claim 1, wherein the shell comprises an additional shell-forming base material in an amount of 100 parts by mass or less relative to 100 parts by mass of the gellan gum.

11. The capsule according to claim 1, wherein the shell comprises a plasticizer.

12. The capsule according to claim 1, wherein the shell comprises at least one plasticizer selected from polyhydric alcohols, sugar alcohols, disaccharides, polysaccharides, and derivatives thereof

13. The capsule according to claim 1, wherein the capsule has an outer diameter of 0.1 to 15 mm.

14. The capsule according to claim 1, wherein the percentage of the shell in the capsule is 3% by mass or more.

15. The capsule according to claim 1, wherein the capsule has a content, and wherein the percentage of the shell in the capsule is 3 to 50% by mass.

16. The capsule according to claim 1, wherein the capsule has a crush strength with a standard deviation (SD) value of 500 g or less, and wherein the capsule has a crush deformation with a standard deviation (SD) value of 1 mm or less.

17. The capsule according to claim 1, wherein the capsule is a seamless capsule.

18. A method for producing the capsule according to claim 1, the method comprising at least the steps of:
forming a capsule by a drop method in such a manner that the capsule has a water content of 80% by mass or more and that the capsule has a ratio of a crush strength (g) to an outer diameter (mm) (crush strength/outer diameter ratio) of 5.0 or more; and
drying the capsule obtained in the capsule formation step.
